(19) United States
(12) Patent Application Publication
(10) Pub. No.: US 2025/0241716 A1
(43) Pub. Date: Jul. 31, 2025

(54) ENDOSCOPY VIDEO TIMELINE INTEREST LEVEL PREDICTION

(71) Applicant: GYRUS ACMI, INC. D/B/A OLYMPUS SURGICAL TECHNOLOGIES AMERICA, Westborough, MA (US)

(72) Inventors: Thorsten Juergens, Hamburg (DE); Michael Ryan, Blaine, MN (US)

(21) Appl. No.: 19/041,298

(22) Filed: Jan. 30, 2025

Related U.S. Application Data
(60) Provisional application No. 63/626,778, filed on Jan. 30, 2024.

(51) Int. Cl. A61B 34/00; G06T 7/00; H04N 19/30

(52) U.S. Cl. CPC A61B 34/25; G06T 7/0012; H04N 19/30; G06T 2200/24; G06T 2207/10016; G06T 2207/20081; G06T 2207/30004

(57) ABSTRACT

Various techniques are described for analyzing video recordings for endoscopic and other types of medical imaging. In some examples, a system generates an intelligent interest prediction indicator displayed in association with a timeline search bar of the video recording. The intelligent interest prediction indicator is quantified and scored over the timeline based on one or more parameters. In other examples, a system intelligently selects key frames from different video segments to display as thumbnails associated with those segments. These techniques may increase the efficiency, accuracy, and speed with which a physician may review and identify salient aspects of a recorded endoscopy procedure.

### PRIORITY CLAIM

This application claims the benefit of priority to U.S. Provisional Patent Application Ser. No. 63/626,778, filed Jan. 30, 2024, the contents of which are hereby incorporated by reference.

### FIELD OF THE DISCLOSURE

This document pertains generally, but not by way of limitation, to medical imaging systems and, more particularly to endoscopy systems.

### BACKGROUND

Endoscopy is a medical procedure that allows physicians to view the inside of the body without making large incisions. This procedure involves the use of an endoscope, a flexible tube with a light and camera attached to it. The endoscope may be inserted through natural openings of the body, such as the mouth, or through small incisions. Endoscopy is used for various purposes, including diagnosing and treating conditions within the gastrointestinal tract, respiratory system, and other organs. It is a minimally invasive method that provides a detailed view of the internal organs, allowing for more accurate diagnosis and targeted treatments. Common types of endoscopic procedures include gastroscopy, for examining the upper digestive tract, colonoscopy for the lower bowel, and bronchoscopy for the lungs.

Endoscopic procedures are highly beneficial in modern medicine due to their minimally invasive nature, leading to reduced recovery time and lower risk of complications compared to traditional surgeries. These procedures are generally safe and are performed under local or general anesthesia to ensure patient comfort. Physicians may not only diagnose conditions through endoscopy but also perform various treatments, such as biopsy, polyp removal, and even some forms of surgery, directly through the endoscope. This versatility makes endoscopy an essential tool in many areas of medicine, including gastroenterology, pulmonology, and oncology. Advances in endoscopic technology continue to enhance its effectiveness, offering high-resolution images and new techniques for treatment and diagnosis.

### SUMMARY OF THE DISCLOSURE

This disclosure describes various techniques for analyzing video recordings for endoscopic and other types of medical imaging. In some examples, a system generates an intelligent interest prediction indicator displayed in association with a timeline search bar of the video recording. The intelligent interest prediction indicator is quantified and scored over the timeline based on one or more parameters. In other examples, a system intelligently selects key frames from different video segments to display as thumbnails associated with those segments. These techniques may increase the efficiency, accuracy, and speed with which a physician may review and identify salient aspects of a recorded endoscopy procedure.

In some aspects, this disclosure is directed to a system for navigating frames of a segment of a video recording of a medical procedure, the system comprising: a user interface including a display; and a processing unit configured for: selecting, for the segment, a thumbnail image based on an assessment of potential interest; displaying, on the user interface, the selected thumbnail image; and receiving, on the user interface, input from a user that selects the displayed thumbnail image.

In some aspects, this disclosure is directed to a system for navigating frames of a segment of a video recording of a medical procedure, the system comprising: a user interface including a display; and a processing unit configured for: determining, based on an assessment of potential interest and a current selection of one or more user-selectable parameters, a prediction indicator for the segment of the video recording; displaying, on the user interface, the prediction indicator; and aligning the prediction indicator with a timeline of the segment.

In some aspects, this disclosure is directed to a system for navigating frames of a segment of a video recording of a medical procedure, the system comprising: a processing unit configured for: determining a prediction indicator for the segment of the video recording based on an assessment of potential interest; dynamically adjusting a compression rate of the segment based on the prediction indictor; and storing the video recording at the adjusted compression rate.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a schematic diagram of an example of an endoscopy system comprising an imaging and control system and an endoscope.

FIG. 2 is a schematic diagram of the endoscopy system of FIG. 1 comprising the endoscope connected to a control unit of the imaging and control system.

FIG. 3 depicts an example of a frame of a segment of a video recording of a medical procedure and a corresponding thumbnail image within the segment from the video recording.

FIG. 4A illustrates an example of a graphical display of a prediction indictor displayed along with various user-selectable parameters.

FIG. 4B illustrates another example of a graphical display of a prediction indictor displayed along with various user-selectable parameters.

FIG. 5 is a schematic diagram of an example of a computer-based tissue sample analyzer.

FIG. 6 shows a schematic diagram of an example of a trained machine learning model.

FIG. 7 depicts a flow diagram of an example of a method for navigating frames of a segment of a video recording of a medical procedure.

FIG. 8 depicts a flow diagram of an example of a method for navigating frames of a segment of a video recording of a medical procedure.

FIG. 9 depicts a flow diagram of an example of a method for navigating frames of a segment of a video recording of a medical procedure.

FIG. 10 is a block diagram illustrating an example of a machine upon which one or more examples may be implemented.

### DETAILED DESCRIPTION

Endoscopy procedures are important diagnostic and therapeutic tools in the medical field. Advances in endoscope devices and imaging technology have enabled high-quality video capture during endoscopic examinations. However, increasing video volumes pose a challenge for physicians to efficiently review and analyze the recorded footage.

Manually scrolling through these large video files to identify salient portions may be time-consuming and inefficient. Artificial intelligence and machine learning algorithms have shown promise in automated analysis of medical images and video. However, techniques to integrate these algorithms to aid physicians in quickly navigating recorded endoscopy videos are lacking.

Therefore, the present inventors have recognized a need for improved graphical user interface systems and methods that leverage analysis of endoscopic video content to assist physicians in readily locating important video segments and key images. Intelligent analytics coupled with dynamic interfaces may aid physician workflow and improve patient care.

This disclosure describes various techniques for analyzing video recordings for endoscopic and other types of medical imaging. In some examples, a system generates an intelligent interest prediction indicator displayed in association with a timeline search bar of the video recording. The intelligent interest prediction indicator is quantified and scored over the timeline based on one or more parameters. In other examples, a system intelligently selects key frames from different video segments to display as thumbnails associated with those segments. These techniques may increase the efficiency, accuracy, and speed with which a physician may review and identify salient aspects of a recorded endoscopy procedure.

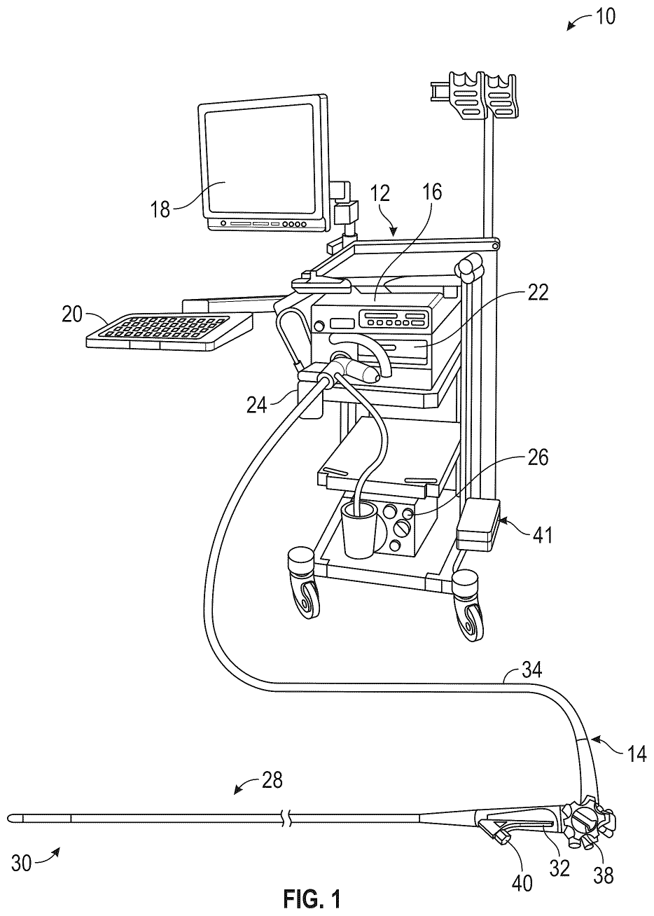

FIG. 1 is a schematic diagram of an example of an endoscopy system 10 comprising an imaging and control system 12 and an endoscope 14. The endoscopy system 10 is suitable for use with the systems, devices, and methods described below, such as modular endoscopy systems, modular endoscopes, and methods for designing, building, and deconstructing endoscopes. According to some examples, the endoscope 14 may be insertable into an anatomical region for imaging and/or to provide passage of one or more sampling devices for biopsies, or one or more therapeutic devices for treatment of a disease state associated with the anatomical region. Endoscope 14 may, in advantageous aspects, interface with and connect to imaging and control system 12. In the example shown, the endoscope 14 includes a colonoscope, though other types of endoscopes may be used with the features and teachings of the present disclosure.

The imaging and control system 12 may include a controller 16, a user interface including an output unit 18 (e.g., display) and an input unit 20, a light source 22, a fluid source 24, and a suction pump 26. The imaging and control system 12 may include various ports for coupling with the endoscopy system 10. For example, the controller 16 may include a data input/output port for receiving data from and communicating data to the endoscope 14.

The light source 22 may include an output port for transmitting light to the endoscope 14, such as via a fiber optic link. The fluid source 24 may include a port for transmitting fluid to the endoscope 14. The fluid source 24 may comprise a pump and a tank of fluid or may be connected to an external tank, vessel, or storage unit. The suction pump 26 may include a port used to draw a vacuum from the endoscope 14 to generate suction, such as for withdrawing fluid from the anatomical region into which the endoscope 14 is inserted. The output unit 18 and the input unit 20 may be used by an operator of the endoscopy system 10 to control functions of the endoscopy system 10 and view the output of the endoscope 14.

The controller 16 may additionally be used to generate signals or other outputs from treating the anatomical region into which the endoscope 14 is inserted. In some examples, the controller 16 may generate electrical output, acoustic output, a fluid output, and the like for treating the anatomical region with, for example, cauterizing, cutting, freezing, and the like.

The endoscope 14 may include an insertion section 28, a functional section 30, and a handle section 32, which may be coupled to a cable section 34 and a coupler section 36. The insertion section 28 may extend distally from the handle section 32 and the cable section 34 may extend proximally from the handle section 32. The insertion section 28 may be elongated and include a bending section, and a distal end to which functional section 30 may be attached. The bending section may be controllable (e.g., by a control knob 38 on a handle section 32) to maneuver the distal end through tortuous anatomical passageways (e.g., stomach, duodenum, kidney, ureter, etc.) The insertion section 28 may also include one or more working channels (e.g., an internal lumen) that may be elongated and support the insertion of one or more therapeutic tools of the functional section 30. The working channel may extend between the handle section 32 and the functional section 30. Additional functionalities, such as fluid passages, guide wires, and pull wires may also be provided by the insertion section 28 (e.g., via suction or irrigation passageways, and the like).

The handle module 32 may include a knob 38 as well as ports 40. The knob 38 may be coupled to a pull wire extending through the insertion section 28. The ports 40 may be configured to couple various electrical cables, fluid tubes, and the like to the handle module 32 for coupling with the insertion section 28.

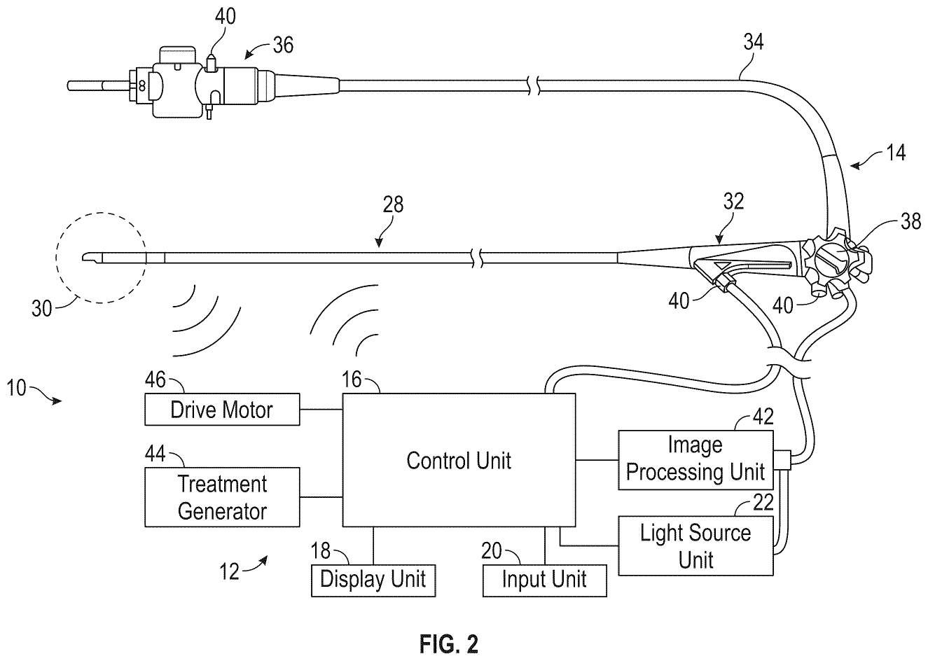

The imaging and control system 12, according to some examples, may be provided on a mobile platform (e.g., a cart 41) with shelves for housing the light source 22, the suction pump 26, the image processing unit 42, etc. Alternatively, several components of the imaging and control system 12 that are shown in FIGS. 1 and 2 may be provided directly on the endoscope 14 so as to make the endoscope “self-contained.”

FIG. 2 is a schematic diagram of the endoscopy system 10 of FIG. 1 including the imaging and control system 12 and endoscope 14. FIG. 2 schematically illustrates components of the imaging and control system 12 coupled to the endoscope 14, which in the illustrated example includes a colonoscope. The imaging and control system 12 may include the controller 16, which may include or be coupled to the image processing unit 42, the treatment generator 44 and the drive unit 46, as well as the light source 22, the input unit 20, and the output unit 18. The image processing unit 42 includes one or more processors that may be distributed, such as locally and remotely, or co-located at one location.

The image processing unit 42 and the light source 22 may each interface with the endoscope 14 by wired or wireless electrical connections. The imaging and control system 12 may accordingly illuminate an anatomical region, collect signals representing the anatomical region, process signals representing the anatomical region, and display images representing the anatomical region on the display unit 18. The imaging and control system 12 may include the light source 22 to illuminate the anatomical region using light of desired spectrum (e.g., broadband white light, narrow-band imaging using preferred electromagnetic wavelengths, and the like). The imaging and control system 12 may connect (e.g., via an endoscope connector) to the endoscope 14 for signal transmission (e.g., light output from light source, video signals from imaging system in the distal end, and the like).

The fluid source 24 may include one or more sources of air, saline or other fluids, as well as associated fluid pathways (e.g., air channels, irrigation channels, suction channels) and connectors (barb fittings, fluid seals, valves and the like). The imaging and control system 12 may also include the drive unit 46, which may be an optional component. The drive unit 46 may include a motorized drive for advancing a distal section of endoscope 14, as described in PCT Pub. No. WO 2011/140118 A1 to Frassica et al., titled “Rotate-to-Advance Catheterization System,” which is hereby incorporated in its entirety by reference.

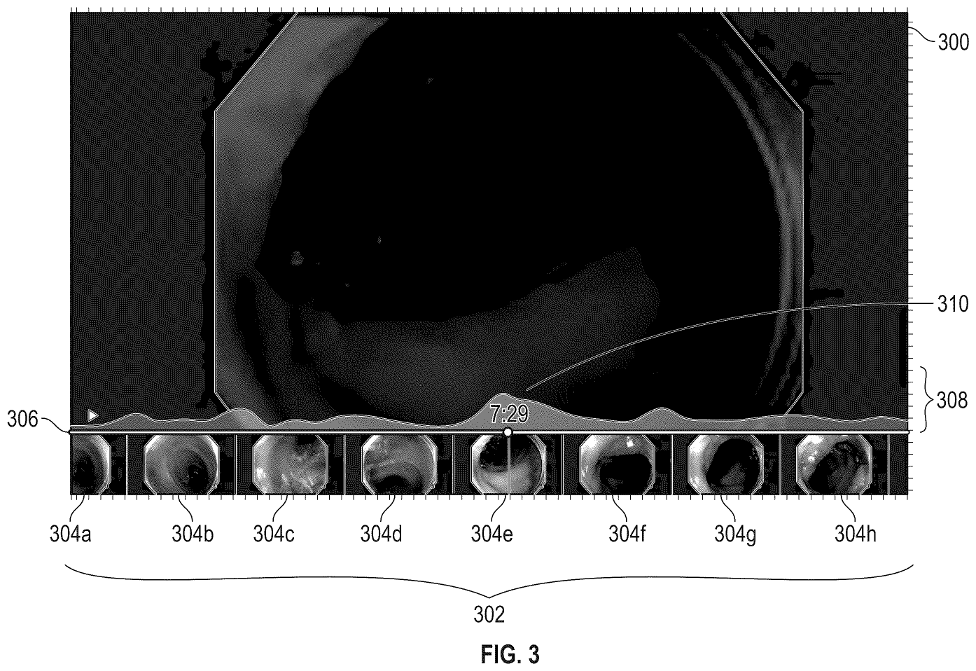

FIG. 3 depicts an example of an image 300 of a segment of a video recording of a medical procedure. As mentioned above, this disclosure describes a system that intelligently selects key frames from different video segments to display as thumbnails associated with those segments. These techniques may increase the efficiency, accuracy, and speed with which a physician may review and identify salient aspects of a recorded endoscopy procedure.

A processing unit of a system, such as the image processing unit 42 of the endoscopy system 10 of FIG. 2 or another processing unit not associate with the endoscopy system 10, may divide a video recording of a medical procedure into a plurality of segments, such as eight segments in a non-limiting example. Then, based on an assessment of potential interest, the processing unit may select, for each segment, a thumbnail image.

In some examples, the assessment of potential interest is determined based on an analysis of the video recording. As an example, the analysis of the video recording includes a detection score for various parameters. These parameters may include: disease detection (e.g. CADe (Computer-Aided Detection) and CADx (Computer-Aided Diagnosis) output) and a potential associated confidence level; activation of light imaging modes at various stages (e.g. white light, narrow band imaging (NBI)); endoscope withdrawal speed; bowel cleanliness score (which may be determined by mucosa detection rate); detection of a tool in the image; detection of blood in the image; and detection of a foreign object in the image. In this manner, the processing unit may intelligently select key frames from different video segments of a video recording of a medical procedure.

CADe involves algorithms or systems designed to assist radiologists or other medical professionals by highlighting areas of interest in medical images. These areas might indicate the presence of abnormalities such as tumors, fractures, or other pathological changes. CADe systems do not make a diagnosis; they simply draw attention to regions that may require further analysis. CADx systems go a step further by not only detecting abnormalities but also providing an interpretation or a possible diagnosis. These systems analyze medical images and offer diagnostic suggestions based on the visual patterns they detect. CADx systems may be used to assist in the decision-making process by providing additional information or a second opinion to the radiologist or physician.

Based on the assessment of potential interest for each corresponding segment, the processing unit selects, for each of the segments, a corresponding thumbnail image, shown in thumbnail ribbon 302 as thumbnail image 304a through thumbnail image 304h. The system displays the selected thumbnail image 304a through thumbnail image 304h, such as on the user interface, e.g., display 18 of FIG. 1 or on another display that is not associated with the endoscopy system 10, such as a personal computing device or tablet computing device. In some examples, the selected thumbnail images are displayed along a timeline 306 of the video recording. In this manner, the clinician knows from where in the video recording of the medical procedure the selected thumbnail image occurred.

Next, via the user interface, such as the input unit 20 of FIG. 1, the system receives input from a user that selects the displayed thumbnail image. For example, in FIG. 3, a user has selected the displayed thumbnail image 304e, which the processing unit identified as a key frame and which is displayed above as larger image 300. If the user selects another key frame that the processing unit selected from a segment, such as the thumbnail image 304b, then the thumbnail image 304b will be displayed above as larger image 300, thereby enabling the user to navigate between segments by selecting the displayed thumbnail images.

As mentioned above, in some examples, the system generates an intelligent interest prediction indictor 308. A processing unit of a system, such as the image processing unit 42 of the endoscopy system 10 of FIG. 2, may determine a prediction indictor 308 for a segment of the video recording based on an assessment of potential interest.

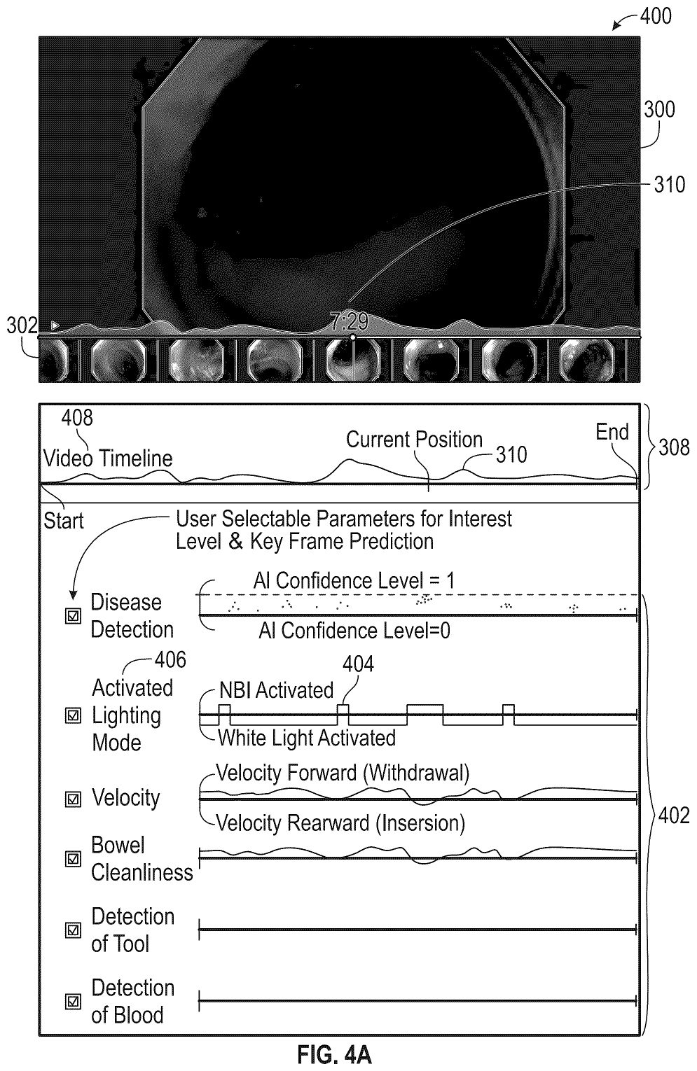

In some examples, the assessment of potential interest is determined based on an analysis of the video recording. The intelligent interest prediction indicator is quantified and scored over the timeline based on one or more parameters. As an example, the analysis of the video recording includes a detection score for various parameters. Like above, in some examples, the analysis of the video recording includes a detection score for one or more of the following parameters: presence of disease; activation of light imaging modes; scope velocity; bowel cleanliness; presence of tools; presence of foreign object; and presence of blood. In some examples, such as shown in detail in FIG. 4A, the parameters are user-selectable.

The processing unit may then display the prediction indictor 308 on the user interface, such as the display 18 of FIG. 1. The prediction indictor 308 includes a line 310 having peaks and troughs, where peaks represent higher levels of assessed potential interest, and troughs represent lower levels of assessed potential interest. The processing unit may align the prediction indictor 308 with a timeline 306 of the video recording.

In addition, the thumbnail images may be aligned with the prediction indictor 308. For example, the thumbnail image 304e is shown aligned with the highest peak of the prediction indictor 308. A user, e.g., clinician, after seeing the prediction indictor 308 may select the thumbnail image 304e to further review that segment of the video recording.

In some examples, the processing unit is configured for dynamically adjusting a compression rate of the segment based on the prediction indictor, and storing the video recording at the adjusted compression rate. For example, it may be desirable to increase the compression rate for segments having a prediction indicator less than a threshold value, which may allow the system to store more data when assessed to be of less interest. In some examples, the system may increase the compression rate for segments having the prediction indicator less than the threshold value after a configurable period of time has elapsed. Similarly, it may be desirable to decrease the compression rate for segments having a prediction indicator greater than a threshold value, which may allow the system to improve the quality of the stored video recording by storing more data.

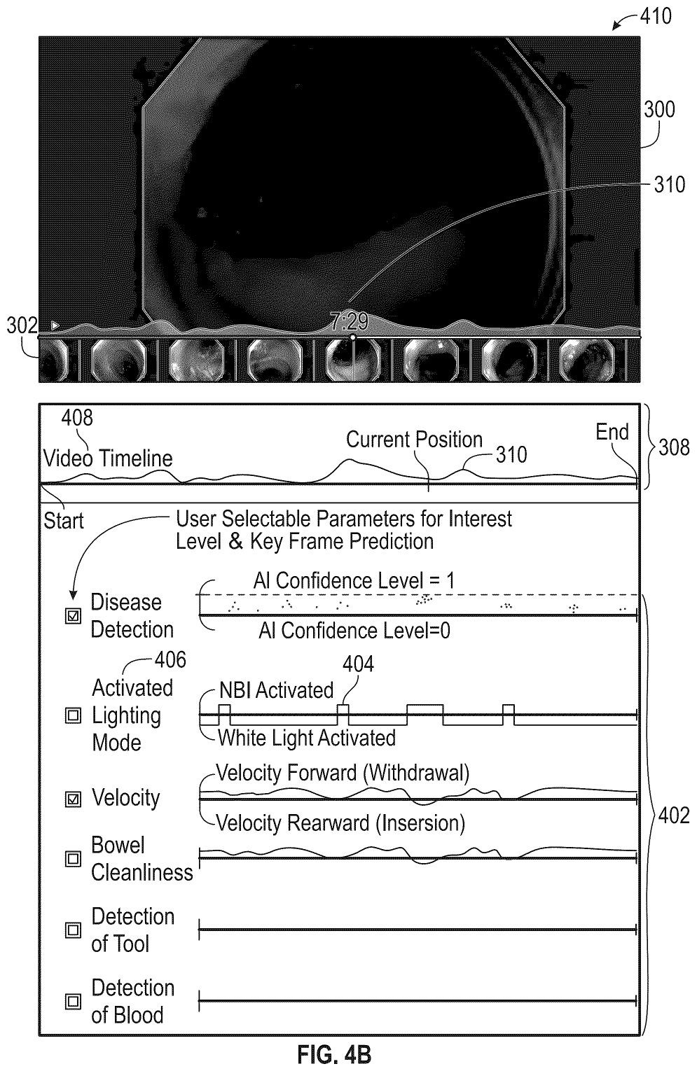

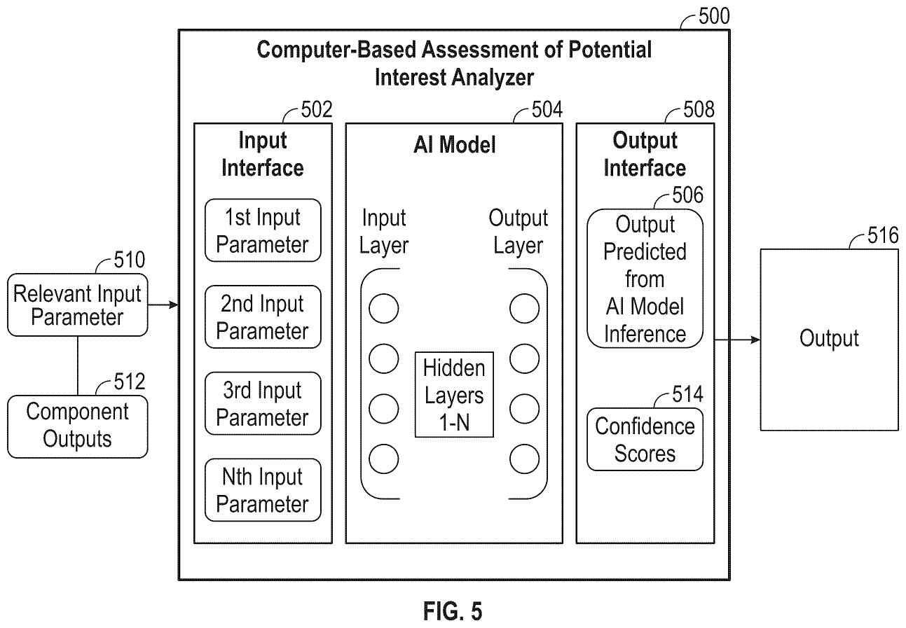

In some examples, the assessment of potential interest for the intelligent key frame selection and/or the intelligent interest prediction indictor is determined based on a trained machine learning model, such as described below with respect to FIG. 5 and FIG. 6. In some such examples, the trained machine learning model is trained using past behavior of a clinician.

It should be noted that, in other examples, the analysis of the video recording need not be performed by the endoscopy system 10. Instead, the video recordings may be stored on a mass-storage device outside the endoscopy system 10, such as on a central server in the hospital or on a remotely-located computing device, e.g., a cloud-based computing device. The techniques of this disclosure may then be used and the results may be displayed on a user interface not associated with the endoscopy system, such as a tablet, laptop computer, desktop computer, or some other device with a display.

FIG. 4A illustrates an example of a graphical display 400 of a prediction indictor 308 displayed along with various user-selectable parameters 402. The processing unit may generate the graphical display 400 on a user interface, such as the display 18 of FIG. 1.

The parameters used by the processing unit to determine the assessment of potential interest may be user-selectable to dynamically update the intelligent interest prediction indicator score based only on a currently selected subset of available parameters. In this manner, if a user recalls a detection of blood and desires to review the associated video, the user can de-select all parameters except the detection of blood. This may immediately draw the user's attention to the specific portion of the recording showing the blood.

In the example shown in FIG. 4A, the user-selectable parameters 402 are displayed and the data representing the user-selectable parameters is aligned with the timeline of the segment. For example, the data 404 of the activated lighting mode 406 is aligned with the video timeline 408.

FIG. 4B illustrates another example of a graphical display of a prediction indictor displayed along with various user-selectable parameters. FIG. 4B includes features that are similar to those shown and described above with respect to FIG. 4A and similar reference numbers are used for such features. For brevity, those features will not be described again in detail.

In the example shown in FIG. 4A, all of the user-selectable parameters 402 were selected. In contrast, the graphical display 410 shown in FIG. 4B has only a subset of the user-selectable parameters 402 were selected, namely the disease detection parameter and the velocity parameter. Additional or alternative parameters may be selected in other examples. Selecting or deselecting one or more of the user-selectable parameters 402 may dynamically adjust the prediction indictor 308 to indicate where the processing unit, e.g., the image processing unit 42 of FIG. 2, identified anomalies so a physician reviewing the video may easily find these times. For example, dynamic adjustment of the prediction indicator 308 may cause the peaks and troughs of the line 310 to shift, indicating changes in the levels of assessed potential interest associated with particular thumbnail images of the thumbnail ribbon 302. In this manner, the processing unit determines, based on an assessment of potential interest and a current selection of one or more user-selectable parameters, a prediction indicator for the segment of the video recording. Upon receiving a user input that changes the current selection to an updated selection; the processing unit dynamically adjusts the prediction indicator based on the updated selection.

FIG. 5 shows a schematic diagram of an example of a computer-based assessment of potential interest analyzer. The computer-based assessment of potential interest analyzer 500 is configured for, among other things, selecting, for one or more segments, a corresponding thumbnail image based on an assessment of potential interest, such as based on an analysis of the video recording.

In some examples, the computer-based assessment of potential interest analyzer 500 analyzes the video recording and generates a detection score for various parameters. In some examples, the computer-based assessment of potential interest analyzer 500 may include an input interface 502 through which various parameters are provided as input features to a trained machine learning (ML) or artificial intelligence (AI) model, such as trained AI model 504. One or more relevant input parameters 510, which may be extracted from various component outputs 512, are applied to the AI model to generate an output predicted from AI model inference 506. The relevant input parameters 510 may include, but are not limited to one or more of the following parameters: presence of disease; activation of light imaging modes; scope velocity; bowel cleanliness; presence of foreign object; presence of tools; and presence of blood.

For example, one or more relevant input parameters 510, which may be extracted from sensor data generated by component outputs 512 of various components of the endoscopy system 10 the of FIG. 1, may be applied to the trained AI model 504, and the AI model 504 may generate confidence scores 514 for the various parameters. Then, based on an assessment of potential interest, the AI model 504 selects, for a segment, a thumbnail image for display on a user interface.

In other examples, the computer-based assessment of potential interest analyzer 500 determines a prediction indicator for the segment of the video recording based on the assessment of potential interest, such as the prediction indictor 308 of FIG. 3. The processing unit, such as the image processing unit 42 of FIG. 2, displays, on the user interface, the prediction indicator, and aligns the prediction indicator with a timeline of the segment, such as shown in FIG. 4A.

In some embodiments, the input interface 502 may be a direct data link between the computer-based assessment of potential interest analyzer 500 and one or more medical devices (e.g., the endoscopy system 10 of FIG. 1), that generates at least some of the input parameters. Additionally, or alternatively, the input interface 502 may be a classical user interface that facilitates interaction between a user and the computer-based assessment of potential interest analyzer 500. For example, the input interface 502 may facilitate a user interface through which the user may manually enter information.

Based on one or more of the input parameters, the output predicted from AI model inference 506 performs an inference operation using the AI model 504 to generate an assessment of potential interest and select, based on the assessment of potential interest, a corresponding thumbnail image for the segment of the video recording. For example, input interface 502 may deliver the input parameters into an input layer of the AI model 504, which propagates these input parameters through the AI model 504 to an output layer. The AI model 504 may provide a computer system the ability to perform tasks, without explicitly being programmed, by making inferences based on patterns found in the analysis of data. The AI model 504 explores the study and construction of algorithms (e.g., machine-learning algorithms) that may learn from existing data and make predictions about new data. Such algorithms operate by building an AI model from example training data in order to make data-driven predictions or decisions expressed as outputs or assessments.

There are two common modes for machine learning (ML): supervised ML and unsupervised ML. Supervised ML uses prior knowledge (e.g., examples that correlate inputs to outputs or outcomes) to learn the relationships between the inputs and the outputs. The goal of supervised ML is to learn a function that, given some training data, best approximates the relationship between the training inputs and outputs so that the ML model may implement the same relationships when given inputs to generate the corresponding outputs. Unsupervised ML is the training of an ML algorithm using information that is neither classified nor labeled, and allowing the algorithm to act on that information without guidance. Unsupervised ML is useful in exploratory analysis because it may automatically identify structure in data.

Common tasks for supervised ML are classification problems and regression problems. Classification problems, also referred to as categorization problems, aim at classifying items into one of several category values (for example, is this object an apple or an orange?). Regression algorithms aim at quantifying some items (for example, by providing a score to the value of some input). Some examples of commonly used supervised-ML algorithms are Logistic Regression (LR), Naive-Bayes, Random Forest (RF), neural networks (NN), deep neural networks (DNN), matrix factorization, and Support Vector Machines (SVM).

Some common tasks for unsupervised ML include clustering, representation learning, and density estimation. Some examples of commonly used unsupervised—ML algorithms are K-means clustering, principal component analysis, and auto-encoders.

Another type of ML is federated learning (also known as collaborative learning) that trains an algorithm across multiple decentralized devices holding local data, without exchanging the data. This approach stands in contrast to traditional centralized machine-learning techniques where all the local datasets are uploaded to one server, as well as to more classical decentralized approaches which often assume that local data samples are identically distributed. Federated learning enables multiple actors to build a common, robust machine learning model without sharing data, thus allowing it to address critical issues such as data privacy, data security, data access rights and access to heterogeneous data.

In some examples, the AI model may be trained continuously or periodically prior to performance of the inference operation by the output predicted from AI model inference 506. Then, during the inference operation, the patient-specific input features provided to the AI model may be propagated from an input layer, through one or more hidden layers, and ultimately to an output layer.

By using these techniques, a processing unit, such as the image processing unit 42 of FIG. 2, may select, for a segment, a thumbnail image based on an assessment of potential interest, display, on the user interface, the selected thumbnail image, and receive, on the user interface, input from a user that selects the displayed thumbnail image.

In other examples, the processing unit may determine a prediction indicator for the segment of the video recording based on an assessment of potential interest, display, on the user interface, the prediction indicator, and align the prediction indicator with a timeline of the segment.

FIG. 6 shows a schematic diagram of an example of a trained machine learning model 600. One approach for training data to develop the trained machine learning model 600 is to leverage annotated endoscopy video footage. This training data contains sample videos that have been reviewed and annotated by clinical experts to indicate timestamps where various parameters of interest occur. For example, the training videos are annotated to tag segments showing presence of specific disease states, activation of certain imaging modes, changes in scope velocity, bowel cleanliness scores, detection of foreign objects, presence of tools, and presence of blood. The annotations indicate the start time and duration of each of those events of interest within the videos.

By training a machine learning model on a dataset containing these expert-annotated videos, the model learns to predict the likelihood of these various events occurring within new unlabeled endoscopy footage. The trained model outputs a detection score over time for each parameter based on patterns learned from the annotated training data.

The imaging processing unit 42 of FIG. 2 may be used to generate data. For example, the imaging processing unit 42 generates data for one or more of the following parameters: presence of disease; activation of light imaging modes; scope velocity; bowel cleanliness; presence of foreign object; presence of tools; and presence of blood. For example, an imaging device 602, such as the imaging and control system 12 of FIG. 1, generates data, such as blood presence data 604, disease data 606, and velocity data 608. The data is used to generate N sets of video training data 610, such as one or more of disease data N 612, scope velocity data N 614, and blood presence data N 616.

One or more signal processing steps may be performed on the video training data 610, such as sampling, feature extraction, filtering, and the like, before the video training data 610 is ready to be used as training data 618. The training data 618 may include N sets of training data based on the video training data 610. In addition, the training data 618 may include annotation training data 620. For example, the annotation training data 620 may include sets of labeling data N 622 and timestamp data N 624 generated by a medical practitioner 626. The neural network structure 628 may include labels associated with one or more parameters. The timestamp data N 624 includes timestamps where various parameters of interest occur.

The training data 618 is used to train an AI or machine learning model, such as the trained machine learning model 600, e.g., the AI model 504 of FIG. 5. The training data 618 may be applied to a neural network structure 628, such as a DNN, comprising an input layer, one or more hidden layers, and an output layer. The training data 618 and the annotation training data 620 may be fed into the input layer of the neural network structure 628, which propagates the input data or data features through one or more hidden layers to the output layer that outputs weights and bias to form the trained machine learning model 600. The trained machine learning model 600 is able to perform tasks without explicitly being programmed by making inferences based on patterns found in the analysis of data.

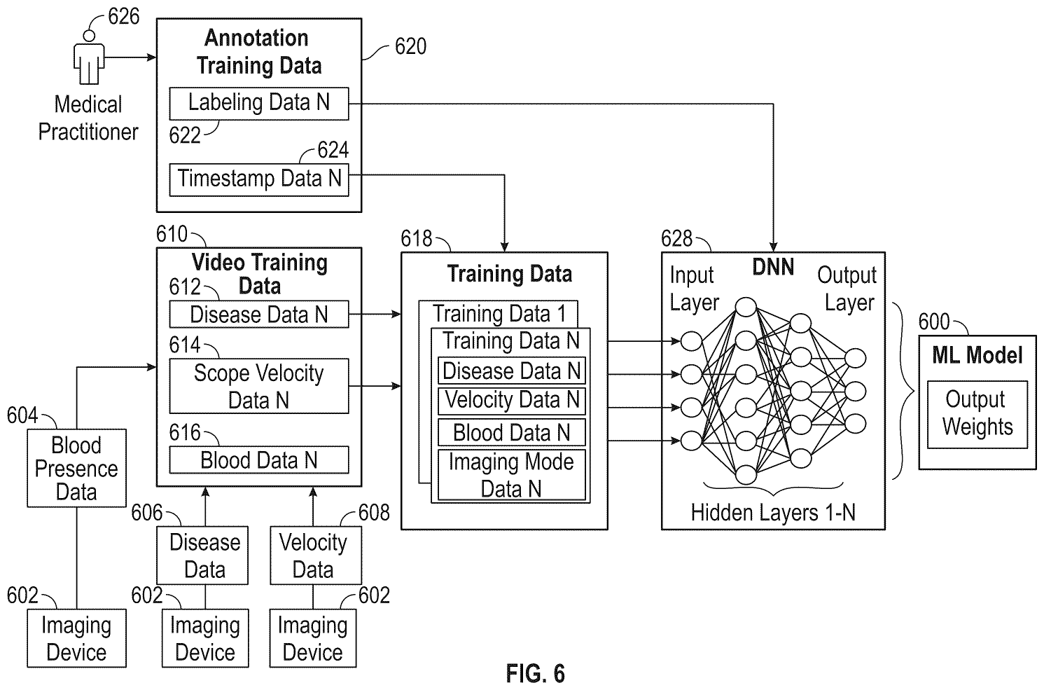

FIG. 7 depicts a flow diagram of an example of a method 700 for navigating frames of a segment of a video recording of a medical procedure. At block 702, the method 700 includes selecting, for a segment, a thumbnail image based on an assessment of potential interest. At block 704, the method 700 includes displaying, on the user interface, the selected thumbnail image. At block 706, the method 700 includes receiving, on the user interface, input from a user that selects the displayed thumbnail image.

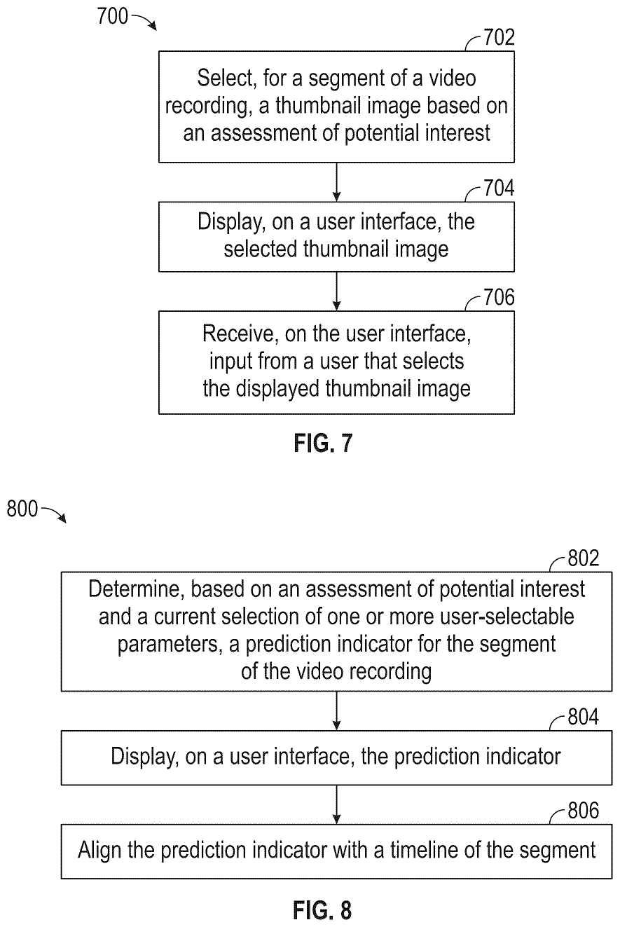

FIG. 8 depicts a flow diagram of an example of a method 800 for navigating frames of a segment of a video recording of a medical procedure. At block 802, the method 800 includes determining, based on an assessment of potential interest and a current selection of one or more user-selectable parameters, a prediction indicator for the segment of the video recording. At block 804, the method 800 includes displaying, on the user interface, the prediction indicator. At block 806, the method 800 includes aligning the prediction indicator with a timeline of the segment.

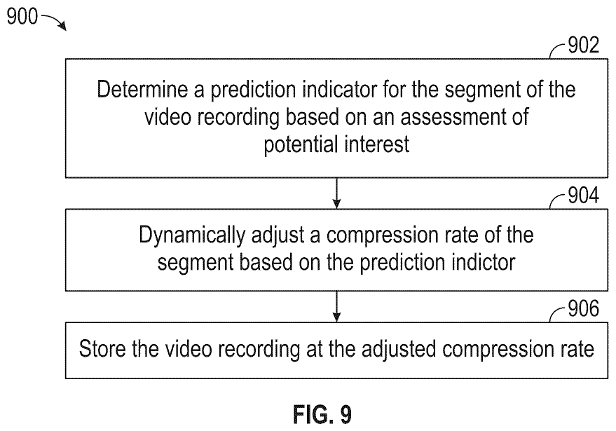

FIG. 9 depicts a flow diagram of an example of a method 900 for navigating frames of a segment of a video recording of a medical procedure. At block 902, the method 900 includes determining a prediction indicator for the segment of the video recording based on an assessment of potential interest. At block 904, the method 900 include dynamically adjusting a compression rate of the segment based on the prediction indictor. At block 906, the method 900 includes storing the video recording at the adjusted compression rate.

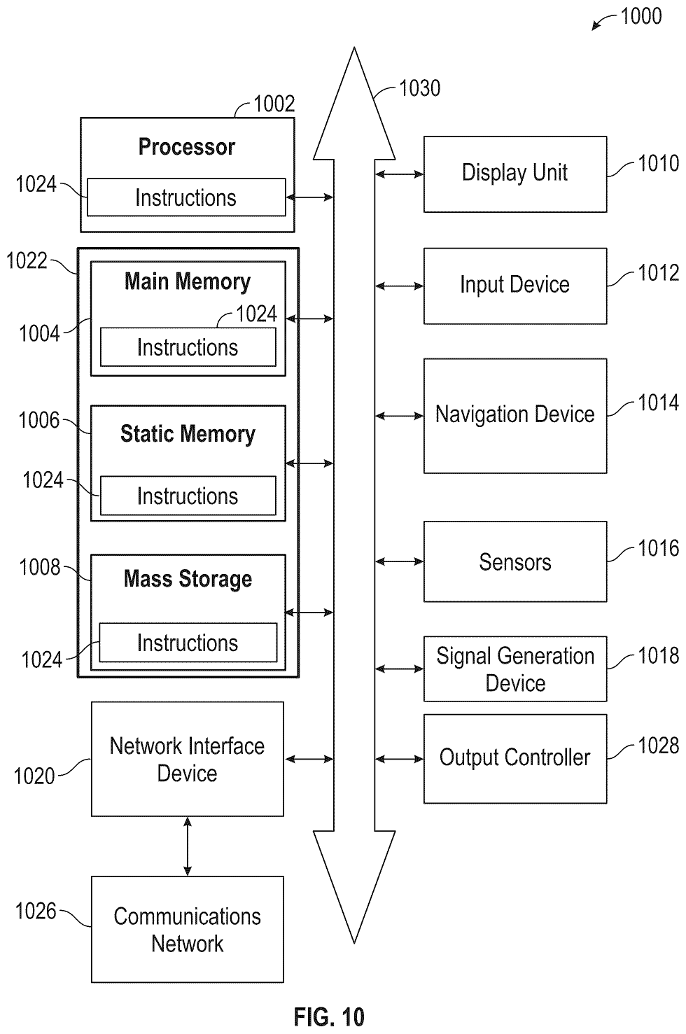

FIG. 10 illustrates a block diagram of an example machine 1000 upon which any one or more of the techniques (e.g., methodologies) discussed herein may perform. Examples, as described herein, may include, or may operate by, logic or a number of components, or mechanisms in the machine 1000. Circuitry (e.g., processing circuitry) is a collection of circuits implemented in tangible entities of the machine 1000 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1000 follow.

In alternative examples, the machine 1000 may operate as a standalone device or may be connected (e.g., networked) to other machines. In a networked deployment, the machine 1000 may operate in the capacity of a server machine, a client machine, or both in server-client network environments. In an example, the machine 1000 may act as a peer machine in peer-to-peer (P2P) (or other distributed) network environment. The machine 1000 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 1000 may include a hardware processor 1002 (e.g., a central processing unit (CPU), a graphics processing unit (GPU), a hardware processor core, or any combination thereof), a main memory 1004, a static memory (e.g., memory or storage for firmware, microcode, a basic-input-output (BIOS), and mass storage 1008 (e.g., hard drives, tape drives, flash storage, or other block devices) some or all of which may communicate with each other via an interlink 530 (e.g., bus). The machine 1000 may further include a display unit 1010, an alphanumeric input device 1012 (e.g., a keyboard), and a user interface (UI) navigation device 1014 (e.g., a mouse). In an example, the display unit 1010, input device 1012 and UI navigation device 1014 may be a touch screen display. The machine 1000 may additionally include a signal generation device 1018 (e.g., a speaker), a network interface device 1020, and one or more sensors 1016, such as a global positioning system (GPS) sensor, compass, accelerometer, or other sensor. The machine 1000 may include an output controller 1028, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 1002, the main memory 1004, the static memory 1006, or the mass storage 1008 may be, or include, a machine readable medium 1022 on which is stored one or more sets of data structures or instructions 1024 (e.g., software) embodying or utilized by any one or more of the techniques or functions described herein. The instructions 1024 may also reside, completely or at least partially, within any of registers of the processor 1002, the main memory 1004, the static memory 1006, or the mass storage 1008 during execution thereof by the machine 1000. In an example, one or any combination of the hardware processor 1002, the main memory 1004, the static memory 1006, or the mass storage 1008 may constitute the machine readable media 1022. While the machine readable medium 1022 is illustrated as a single medium, the term “machine readable medium” may include a single medium or multiple media (e.g., a centralized or distributed database, and/or associated caches and servers) configured to store the one or more instructions 1024.

The term “machine readable medium” may include any medium that is capable of storing, encoding, or carrying instructions for execution by the machine 1000 and that cause the machine 1000 to perform any one or more of the techniques of the present disclosure, or that is capable of storing, encoding or carrying data structures used by or associated with such instructions. Non-limiting machine readable medium examples may include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

In an example, information stored or otherwise provided on the machine readable medium 1022 may be representative of the instructions 1024, such as instructions 1024 themselves or a format from which the instructions 1024 may be derived. This format from which the instructions 1024 may be derived may include source code, encoded instructions (e.g., in compressed or encrypted form), packaged instructions (e.g., split into multiple packages), or the like. The information representative of the instructions 1024 in the machine readable medium 1022 may be processed by processing circuitry into the instructions to implement any of the operations discussed herein. For example, deriving the instructions 1024 from the information (e.g., processing by the processing circuitry) may include: compiling (e.g., from source code, object code, etc.), interpreting, loading, organizing (e.g., dynamically or statically linking), encoding, decoding, encrypting, unencrypting, packaging, unpackaging, or otherwise manipulating the information into the instructions 1024.

In an example, the derivation of the instructions 1024 may include assembly, compilation, or interpretation of the information (e.g., by the processing circuitry) to create the instructions 1024 from some intermediate or preprocessed format provided by the machine readable medium 1022. The information, when provided in multiple parts, may be combined, unpacked, and modified to create the instructions 1024. For example, the information may be in multiple compressed source code packages (or object code, or binary executable code, etc.) on one or several remote servers. The source code packages may be encrypted when in transit over a network and decrypted, uncompressed, assembled (e.g., linked) if necessary, and compiled or interpreted (e.g., into a library, stand-alone executable etc.) at a local machine, and executed by the local machine.

The instructions 1024 may be further transmitted or received over a communications network 1026 using a transmission medium via the network interface device 1020 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), LoRa/LoRaWAN, or satellite communication networks, mobile telephone networks (e.g., cellular networks such as those complying with 3G, 4G LTE/LTE-A, or 5G standards), Plain Old Telephone (POTS) networks, and wireless data networks (e.g., Institute of Electrical and Electronics Engineers (IEEE) 1002.11 family of standards known as Wi-Fi®, IEEE 1002.15.4 family of standards, peer-to-peer (P2P) networks, among others. In an example, the network interface device 1020 may include one or more physical jacks (e.g., Ethernet, coaxial, or phone jacks) or one or more antennas to connect to the communications network 1026. In an example, the network interface device 1020 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1000, and includes digital or analog communications signals or other intangible medium to facilitate communication of such software. A transmission medium is a machine-readable medium.

### Various Notes

Each of the non-limiting claims or examples described herein may stand on its own, or may be combined in various permutations or combinations with one or more of the other examples.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more claims thereof), either with respect to a particular example (or one or more claims thereof), or with respect to other examples (or one or more claims thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels and are not intended to impose numerical requirements on their objects.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer-readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact discs and digital video discs), magnetic cassettes, memory cards or sticks, random access memories (RAMs), read-only memories (ROMs), and the like.

The above description is intended to be illustrative, and not restrictive. For example, the above-described examples (or one or more claims thereof) may be used in combination with each other. Other embodiments may be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments may be combined with each other in various combinations or permutations. The scope of the invention should be determined with reference to the appended claims, along with the full scope of equivalents to which such claims are entitled.

What is claimed is:

1. A system for navigating frames of a segment of a video recording of a medical procedure, the system comprising:
a user interface including a display; and
a processing unit configured for:
selecting, for the segment, a thumbnail image based on an assessment of potential interest;
displaying, on the user interface, the selected thumbnail image; and
receiving, on the user interface, input from a user that selects the displayed thumbnail image.

2. The system for navigating frames of claim 1, wherein the assessment of potential interest is determined based on an analysis of the video recording.

3. The system for navigating frames of claim 2, wherein the analysis of the video recording includes a detection score for one or more of the following parameters:
presence of disease;
activation of light imaging modes;
scope velocity;
bowel cleanliness;
presence of foreign object;
presence of tools; and
presence of blood.

4. The system for navigating frames of claim 1, wherein the video recording includes a plurality of segments, wherein the processing unit is further configured for:
selecting, for each of the plurality of segments, a corresponding thumbnail image based on an assessment of potential interest for the corresponding segment;
displaying, on the user interface, the selected thumbnail images aligned with their corresponding segment, and
wherein the user interface is configured for:
enabling the user to navigate between segments by selecting the displayed thumbnail images.

5. The system for navigating frames of claim 1, wherein the assessment of potential interest is determined based on a trained machine learning model.

6. The system for navigating frames of claim 5, wherein the trained machine learning model is trained using past behavior of a clinician.

7. A system for navigating frames of a segment of a video recording of a medical procedure, the system comprising:
a user interface including a display; and
a processing unit configured for:
determining, based on an assessment of potential interest and a current selection of one or more user-selectable parameters, a prediction indicator for the segment of the video recording;
displaying, on the user interface, the prediction indicator; and
aligning the prediction indicator with a timeline of the segment.

8. The system for navigating frames of claim 7, wherein the assessment of potential interest is determined based on an analysis of the video recording.

9. The system for navigating frames of claim 8, wherein the analysis of the video recording includes a detection score for one or more of the following user-selectable parameters:
presence of disease;
activation of light imaging modes;
scope velocity;
bowel cleanliness;
presence of tools;
presence of foreign object; and
presence of blood.

10. The system for navigating frames of claim 9, wherein the processing unit is configured for:
displaying, on the user interface, data representing the user-selectable parameters; and
aligning the data representing the user-selectable parameters with the timeline of the segment.

11. The system for navigating frames of claim 7, wherein the prediction indicator includes a line having peaks and troughs, wherein peaks represent higher levels of assessed potential interest, and troughs represent lower levels of assessed potential interest.

12. The system for navigating frames of claim 7, wherein the processing unit is configured for:
dynamically adjusting a compression rate of the segment based on the prediction indictor; and
storing the video recording at the adjusted compression rate.

13. The system for navigating frames of claim 12, wherein the processing unit is configured for:
increasing the compression rate for segments having a prediction indicator less than a threshold value.

14. The system for navigating frames of claim 7, wherein the assessment of potential interest is determined based on a trained machine learning model.

15. The system for navigating frames of claim 14, wherein the trained machine learning model is trained using past behavior of a clinician.

16. The system for navigating frames of claim 7, wherein the processing unit is configured for:
receiving a user input that changes the current selection to an updated selection; and
dynamically adjusting the prediction indicator based on the updated selection.

17. A system for navigating frames of a segment of a video recording of a medical procedure, the system comprising:
a processing unit configured for:
determining a prediction indicator for the segment of the video recording based on an assessment of potential interest;
dynamically adjusting a compression rate of the segment based on the prediction indictor; and
storing the video recording at the adjusted compression rate.

18. The system for navigating frames of claim 17, wherein the processing unit is configured for:
increasing the compression rate for segments having a prediction indicator less than a threshold value.

19. The system for navigating frames of claim 18, wherein the processing unit configured for increasing the compression rate for segments having a prediction indicator less than a threshold value is configured for:
increasing the compression rate for segments having the prediction indicator less than the threshold value after a configurable period of time has elapsed.

20. The system for navigating frames of claim 17, wherein the assessment of potential interest is determined based on an analysis of the video recording.

21. The system for navigating frames of claim 20, wherein the analysis of the video recording includes a detection score for one or more of the following parameters:
presence of disease;
activation of light imaging modes;
scope velocity;
bowel cleanliness;
presence of foreign object;
presence of tools; and
presence of blood.
